(19) United States
(12) Patent Application Publication
(10) Pub. No.: US 2026/0196335 A1
(43) Pub. Date: Jul. 9, 2026

(54) SPECTRAL CLUSTERING FOR DETECTION OF ATYPICAL CARDIAC CORONARIES

(71) Applicant: KONINKLIJKE PHILIPS N.V., EINDHOVEN (NL)

(72) Inventors: RAFAEL WIEMKER, KISDORF (DE); HANNES NICKISCH, HAMBURG (DE); AXEL SAALBACH, HAMBURG (DE); CLAAS BONTUS, HAMBURG (DE); HARALD SEPP HEESE, HAMBURG (DE); JOSE ALEJANDRO MATUTE FLORES, HAMBURG (DE); JOCHEN PETERS, NORDERSTEDT (DE)

(21) Appl. No.: 19/130,041

(22) PCT Filed: Nov. 16, 2023
(86) PCT No.: PCT/EP2023/081980

§ 371 Date: May 14, 2025

Related U.S. Application Data
(60) Provisional application No. 63/428,517, filed on Nov. 29, 2022.

(51) Int. Cl. G16H 30/20

(52) U.S. Cl. CPC G16H 30/20

(57) ABSTRACT

A method of spectral clustering includes: obtaining a set of coronary image data comprising voxels of a volume of interest around a heart; processing the voxels to rank the voxels for likelihood of characterizing a vessel; identifying similarities between pairs of voxels to quantify the strength of a link between the voxels in each pair of voxels; spectral clustering the voxels based on the likelihood of characterizing a vessel and the strength of links between the pairs of voxels; selecting at least one subtree of voxels based on the spectral clustering; classifying each subtree based on the set of coronary image data; and reconstructing a representation of at least one vessel in the volume of interest as a first reconstruction to include at least one subtree classified based on the set of coronary image data.

### BACKGROUND

Appraisal and quantification of coronary vessels on diagnostic and/or pre-operative computerized tomography (CT) image volumes is regularly performed and requires at least semi-automated segmentation of the coronary vessels. Most anatomical constellations of coronary vessels are characterizable by type, and current appraisal and quantification of such coronary vessels is by type. Standard machine learning models can be trained on conventional computerized tomography scans of known types of constellations of coronary vessels in large image and annotation databases. An inability to identify, segment and develop atypical anatomical constellations may result in an inability to diagnose and treat some cardiac events.

However, atypical coronary constellation assessment are not widely available in current image and annotation databases, let alone curated and annotated. Additionally, spectral and photon-counting computerized tomography scans with improved spectral and/or spatial resolution are not yet widely available in such databases, let alone curated or annotated for atypical coronary constellation assessment.

There is a need for segmentation of atypical constellations of coronary vessels. Such atypical constellations of coronary vessels include, for example, collaterals, by-passes and coronary artery bypass grafts (CABGs).

### SUMMARY

According to an aspect of the present disclosure, a method of spectral clustering includes: obtaining a set of coronary image data comprising voxels of a volume of interest around a heart; processing the voxels to rank the voxels for likelihood of characterizing a vessel; identifying similarities between pairs of voxels to quantify the strength of a link between the voxels in each pair of voxels; spectral clustering the voxels based on the likelihood of characterizing a vessel and the strength of links between the pairs of voxels; selecting at least one subtree of voxels based on the spectral clustering; classifying each subtree based on the set of coronary image data; and reconstructing a representation of at least one vessel in the volume of interest as a first reconstruction to include at least one subtree classified based on the set of coronary image data.

According to another aspect of the present disclosure, a system for spectral clustering includes a memory that stores instructions; and a processor that executes the instructions. When executed by the processor, the instructions cause the system to: obtain a set of coronary image data comprising voxels of a volume of interest around a heart; process the voxels to rank the voxels for likelihood of characterizing a vessel; identify similarities between pairs of voxels to quantify the strength of a link between the voxels in each pair of voxels; spectral cluster the voxels based on the likelihood of characterizing a vessel and the strength of links between the pairs of voxels; select at least one subtree of voxels based on the spectral clustering; classify each subtree based on the set of coronary image data; and reconstruct a representation of at least one vessel in the volume of interest as a first reconstruction to include at least one subtree classified based on the set of coronary image data.

According to another aspect of the present disclosure, a tangible non-transitory computer-readable storage medium stores a computer program. The computer program, when executed by a processor, causes a system to: system for spectral clustering includes a memory that stores instructions; and a processor that executes the instructions. When executed by the processor, the instructions cause the system to: obtain a set of coronary image data comprising voxels of a volume of interest around a heart; process the voxels to rank the voxels for likelihood of characterizing a vessel; identify similarities between pairs of voxels to quantify the strength of a link between the voxels in each pair of voxels; spectral cluster the voxels based on the likelihood of characterizing a vessel and the strength of links between the pairs of voxels; select at least one subtree of voxels based on the spectral clustering; classify each subtree based on the set of coronary image data; and reconstruct a representation of at least one vessel in the volume of interest as a first reconstruction to include at least one subtree classified based on the set of coronary image data.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 illustrates a system for spectral clustering for detection of atypical cardiac coronaries, in accordance with a representative embodiment.

FIG. 2 illustrates a method for spectral clustering for detection of atypical cardiac coronaries, in accordance with a representative embodiment.

FIG. 3 illustrates another method for spectral clustering for detection of atypical cardiac coronaries, in accordance with a representative embodiment.

FIG. 4 illustrates an example of a power iteration for spectral clustering for detection of atypical cardiac coronaries, in accordance with a representative embodiment.

FIG. 5 illustrates an example of projections for spectral clustering for detection of atypical cardiac coronaries, in accordance with a representative embodiment.

FIG. 6A illustrates a chart for a power iteration for spectral clustering for detection of atypical cardiac coronaries, in accordance with a representative embodiment.

FIG. 6B illustrates a chart of maximum power sizes over iterations for the power iteration in FIG. 6A, in accordance with a representative embodiment.

FIG. 7 illustrates an example application on pulmonary vessel trees in spectral clustering for detection of atypical cardiac coronaries, in accordance with a representative embodiment.

FIG. 8 illustrates an example diffusion process of evolving network ran of candidate voxels in spectral clustering for detection of atypical cardiac coronaries, in accordance with a representative embodiment.

FIG. 9 illustrates a computer system, on which a method for spectral clustering for detection of atypical cardiac coronaries is implemented, in accordance with another representative embodiment.

### DETAILED DESCRIPTION

In the following detailed description, for the purposes of explanation and not limitation, representative embodiments disclosing specific details are set forth in order to provide a thorough understanding of embodiments according to the present teachings. However, other embodiments consistent with the present disclosure that depart from specific details disclosed herein remain within the scope of the appended claims. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. Definitions and explanations for terms herein are in addition to the technical and scientific meanings of the terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to”, “coupled to”, or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below.

As described herein, atypical anatomical constellations may be segmented and developed. Additionally, advanced spectral and spatial resolution voxel-features of newly emerging imaging modalities such as spectral and photon-counting computerized tomography may be exploited. Examples of atypical anatomical constellations which may be segmented and developed using newly emerging imaging modalities include, for example, collaterals, by-passes, and coronary artery bypass grafts. Identification, segmentation and development of atypical anatomical constellations may result in an ability to diagnose and treat cardiac events which otherwise would not be diagnosed and treated.

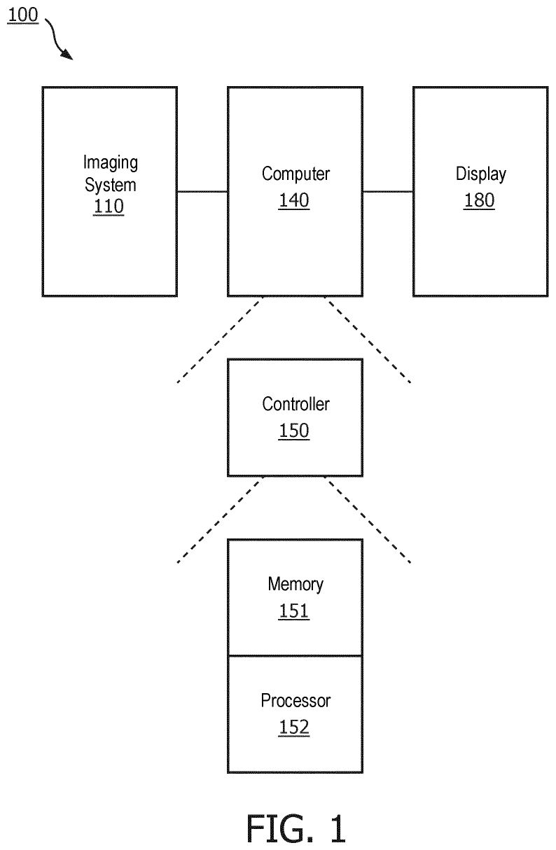

FIG. 1 illustrates a system 100 for spectral clustering for detection of atypical cardiac coronaries, in accordance with a representative embodiment.

The system 100 in FIG. 1 is a system for spectral clustering for detection of atypical cardiac coronaries and includes components that may be provided together or that may be distributed. The system 100 includes an imaging system 110, a computer 140 and a display 180. The computer 140 includes a controller 150, and the controller 150 includes a memory 151 and a processor 152. In some embodiments, the imaging system 110 is provided with the computer 140 and the display 180, such as in a hospital complex or other medical environment. In other embodiments, the imaging system 110 is provided remotely from the computer 140 and the display 180, such as when the computer 140 is representative of a cloud implementation for the functionality described herein and the display 180 is provided in a separate facility.

The imaging system 110 is representative of imaging systems that are used to perform imaging of cardiac coronaries. In descriptions herein, the imaging system 110 is primarily referenced as a computerized tomography imaging system. However, the teachings herein are applicable to all three-dimensional imaging modalities, including computerized tomography enterorrhaphy (CTE), magnetic resonance imaging (MRI) and ultrasound.

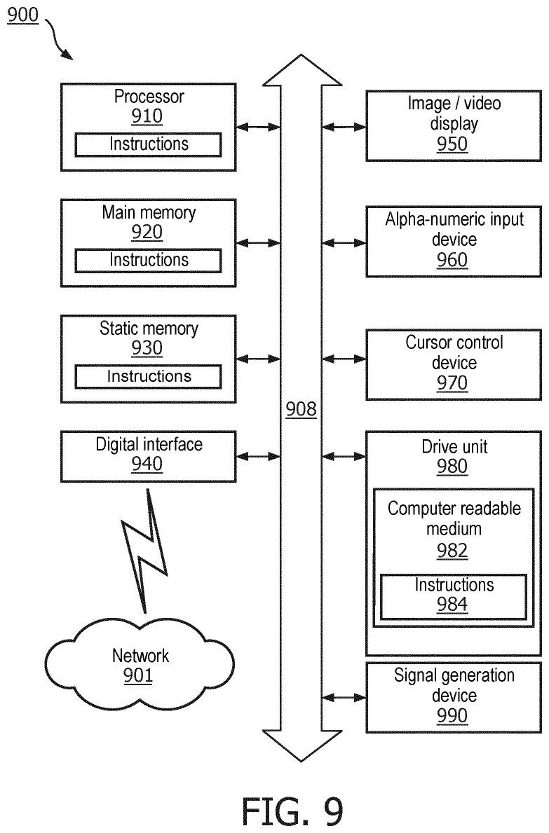

The computer 140 is representative of a desktop or a server implemented in a facility or in the cloud. A computer that can be used to implement the computer 140 is depicted in FIG. 9, though a computer 140 may include more or fewer elements than depicted in FIG. 1 or FIG. 9.

The controller 150 includes at least a memory 151 that stores instructions and a processor 152 that executes the instructions. The controller 150 executes the instructions to perform a method based on coronary image data, such as from computer tomograph images. The method includes obtaining a set of coronary image data comprising voxels of a volume of interest around a heart. That is, the image data is three-dimensional image data comprising voxels of a volume of interest around a heart. The method also includes processing the voxels to rank the voxels for likelihood of characterizing a vessel. The likelihood of characterizing a vessel may be referred to as vesselness insofar as the underlying determination is whether the voxels represent a portion of the volume of interest that includes a vessel. The method also includes identifying similarities between pairs of voxels to quantify the strength of a link between the voxels in each pair of voxels. The method implemented using the controller 150 is looking for pairs of voxels that each represent portions of the volume of interest that include a vessel. The method also includes spectral clustering the voxels based on the likelihood of characterizing a vessel and the strength of links between the pairs of voxels. Voxels that are likely to characterize a vessel may be clustered. The clustering may mean grouping the voxels in two or more groups. The method implemented using the controller 150 also includes selecting at least one subtree of voxels based on the spectral clustering. The subtree may include sets of adjacent voxels that are deemed likely to represent a vessel. The method also includes classifying each subtree based on the set of coronary image data. The method further includes reconstructing a representation of at least one vessel in the volume of interest as a first reconstruction to include at least one subtree classified based on the set of coronary image data.

The display 180 may be local to the computer 140 or may be remotely connected to the computer 140, such as via a local area network or via a wide area network such as the internet. When locally connected, the display 180 may be connected to the computer 140 via a local wired interface such as an Ethernet cable or via a local wireless interface such as a Wi-Fi connection. The display 180 may be interfaced with other user input devices by which users can input instructions, including mouses, keyboards, thumbwheels and so on. The display 180 may be a monitor such as a computer monitor, a display on a mobile device, an augmented reality display, a television, an electronic whiteboard, or another screen configured to display electronic imagery. The display 180 may also include one or more input interface(s) such as those noted above that may connect to other elements or components, as well as an interactive touch screen configured to display prompts to users and collect touch input from users.

The controller 150 may also include interfaces, such as a first interface, a second interface, a third interface, and a fourth interface. One or more of the interfaces may include ports, disk drives, wireless antennas, or other types of receiver circuitry that connect the controller 150 to other electronic elements. One or more of the interfaces may also include user interfaces such as buttons, keys, a mouse, a microphone, a speaker, a display separate from the display 180, or other elements that users can use to interact with the controller 150 such as to enter instructions and receive output.

The controller 150 may perform some of the operations described herein directly and may implement other operations described herein indirectly. For example, the controller 150 may indirectly control operations such as by generating and transmitting content to be displayed on the display 180. The controller 150 may directly control other operations such as logical operations performed by the processor 152 executing instructions from the memory 151 based on input received from electronic elements and/or users via the interfaces. Accordingly, the processes implemented by the controller 150 when the processor 152 executes instructions from the memory 151 may include steps not directly performed by the controller 150.

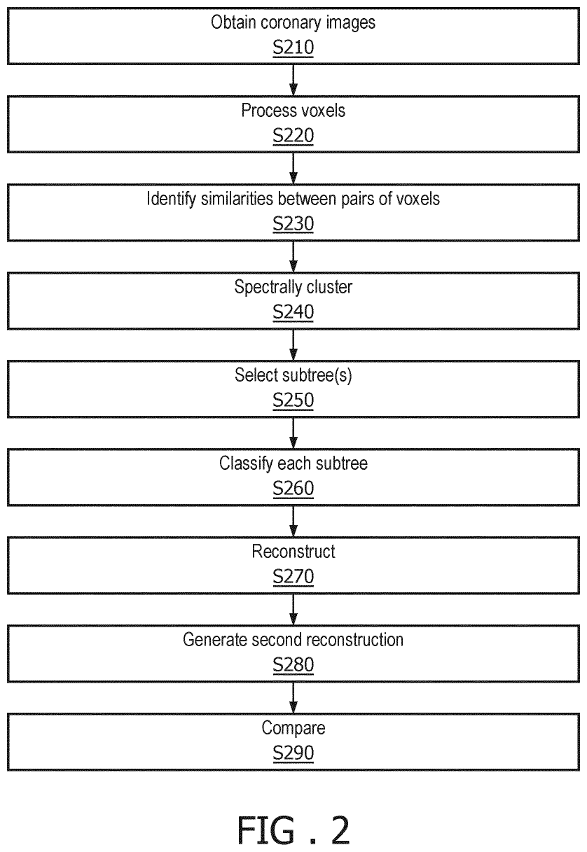

FIG. 2 illustrates a method for spectral clustering for detection of atypical cardiac coronaries, in accordance with a representative embodiment.

The method of FIG. 2 may be performed by the system 100 including the computer 140 with the controller 150. At S210, the method starts by obtaining coronary images. The coronary images consist of coronary image data comprising voxels of a volume of interest around a heart. The coronary images may be received by the computer 140 directly or indirectly from the imaging system 110. For example, the coronary images may be communicated in the same facility such as a hospital complex. Alternatively, the coronary images may be uploaded to the cloud and received at a server in a data center, in which case the server in the data center is the computer 140 performing most of the method in FIG. 2.

At S220, voxels are processed. The method of FIG. 2 is performed to identify and connect vascular structures in a volume of interest around a heart without prior anatomical training. The coronary constellations which are identified and connected in FIG. 2 may include both typical and atypical coronary constellations The voxels are processed at S220 to rank the voxels for likelihood of characterizing a vessel. The processing at S220 may include filtering the voxels based on image properties expected of vessels. The processing of voxels uses power iterations. The likelihood of a voxel characterizing a vessel may be determined based on a vesselness filter applied to the voxel.

At S230, similarities between pairs of voxels are identified. The similarities are identified to quantify the strength of a link between the voxels in each pair of voxels. The similarities are identified based on the image properties expected of vessels. The similarities may be identified based on proximity of coordinates of each voxel in a pair of voxels and directionality of each voxel in the a pair of voxels. The proximity reflects how close the voxels in a pair are to one another, including whether the voxels are adjacent. Directionality reflects whether the voxels are of a vessel flowing in the same direction. For example, similarities may be identified for pairs of proximate voxels and not identified for pairs of remote voxels.

As an example similarity measure, consider s_ij=n∥xᵢ−xⱼ∥/p·k(v_i, v_j)·d(f_i,f_j), where x_i is the integer coordinate of voxel i, ρ is a spatial influence range in voxels e.g., ρ=1, n is a stationary covariance with compact support to quantify the local neighborhood. v_i is the vesselness of voxel i. v_j is the vesselness of voxel j. k is a similarity between the vesselness of voxels i and j. d is the similarity between further image features f_i such as image voxel intensity, similarity in various spectral computerized tomography channels, vector alignment of the local Hessian eigenvectors, vector alignment between local Hessian eigenvector and the node-connecting spatial vector direction, similarity of vesselness from local Hessian eigenvalues, etc.

At S240, the method includes performing spectral clustering. The spectral clustering is performed based on the likelihood of characterizing a vessel and the strength of links between the pairs of vessels. Spectral clustering may be performed based on a matrix of the voxels in the volume of interest and using power iteration clustering. As an example, the more likely two adjacent voxels are to characterize a vessel, the more likely the two adjacent voxels are to be clustered together. However, voxel clustering is not limited to voxels which are adjacent to one another.

As an example of power iteration clustering at S240, the largest eigenvector can be found iteratively by power iterations, making it tractable also for very large matrices. Power iterations can be computed by starting from an initial unit vector when all voxels are of equal rank, then the vector containing all voxel ranks is updated using the graph links and the current weight of all local neighborhood voxels. Voxels which are part of a flow-path emerge with increased rank scores. Voxels at the root of sub-trees emerge with the highest scores, as the confluence location of multiple paths.

In some embodiments, during power iteration towards the random walk convergence state, the rank score s of each voxel may correspond to its entry in the eigenvector and is updated to state s\hat{ }k+1 from the current state s\hat{ }k of its neighbors, using a matrix-vector multiplication. For the sparse matrix L, this may be simplified to a low-computation update of a node score from current neighbor scores. For numerical stability, the state vector may be normalized after each iteration.

In some embodiments, at each iteration state of clustering, the current eigenvector entries can be interpreted as graph node score, and the graph can be clustered into sub-graphs, e.g., using k-means clustering on the scores, graph-mode seeking, or finding connected (linked) components above a certain score threshold.

The processing at S220, the identification of similarities at S230 and the spectral clustering at S240 may be performed on a 1-to-1 basis by cores of a graphical processing unit. For example, the processor 152 may be or include a graphical processing unit with dozens, hundreds or thousands of cores used to process image data for pixels and voxels. The iterative nature of parts of the method of FIG. 2 benefit from parallel implementation such as using graphical processing units or SIMD-capable server central processing units (CPUs), or to being mapped to a hardware-optimized sparse matrix operation.

In some embodiments, parameters used in the identification of similarities may be optimized. Parameters of the similarity measure can be optimized if an annotated training set of images and vessel clusters is available by means of a fidelity criterion such as the mean squared error (MSE) or the mean absolute error (MAE).

In some embodiments, a user may be provided an ability to interactively seed specific vessels as a guide to the approximate computation of the spectral clustering. For example, the ostium of the left anterior descending artery on the aorta may be designated by a user and taken into account to guide the approximate computation of the spectral clustering by means of starting from a non-uniform vector rather than from a uniform or random initialization in the power iteration.

At S250, one or more subtree(s) are selected. The subtree(s) of voxels are selected based on the spectral clustering. The selection of a subtree may involve delineating voxels comprising the subtree and implicitly identifying boundaries of the subtree in three dimensions.

At S260, each selected subtree is classified. The classification at S260 is based on the set of coronary image data. The classification of a subtree may be as a coronary vessel or as a pulmonary vessel. Possible classifications of a subtree may include a typical major coronary artery segment such as LAD, LCD, RCD, Ramus, etc.; an atypical coronary which is an anatomical variant; a coronary vein; a pulmonary vein or artery; an artificial bypass segment; an artificial wire such as a pacemaker-lead; and/or a possible image artifact. The classified subtree may be labeled. Of course, classifications are not limited to the types listed above, and classification may involve more or fewer differentiable types than those listed above.

At S270, the method of FIG. 2 includes reconstructing a vessel as a first reconstruction. Specifically, at S270, a representation of at least one vessel in the volume of interest is reconstructed to include at least one subtree classified based on the set of coronary image data. The reconstructing includes segmenting atypical cardiac coronaries identified by the spectral voxel graph clustering in the method of FIG. 2. The reconstructing may also include segmenting typical cardiac coronaries identified by the spectral voxel graph clustering. The approach of the method of FIG. 2 allows detection and segmentation of coronary constellations which are not easily learned by standard machine learning techniques. Examples of such coronary constellations include coronary constellations for patients after surgical remodeling, or after artificial by-passes.

At S280, a second reconstruction is generated. The second reconstruction may be performed independent of the performance of S220 to S270, and instead based on performing a conventional analysis of typical cardiac coronaries. That is, the second reconstruction in the volume of interest may be performed without performing the spectral clustering and selecting. The conventional analysis may include applying a trained artificial intelligence model to identify predetermined types of vessels.

At S290, a comparison is made between the first reconstruction and the second reconstruction. The comparison at S290 may be performed to identify any atypical coronary constellations from subtree(s) of voxels selected at S260 based on the spectral clustering. Insofar as the second reconstruction should result in a conventional segmentation of coronary vessels in the volume of interest, the first reconstruction may comparatively show atypical coronary vessels. The analytical algorithm described by the method of FIG. 2 may be run in addition to a machine learning algorithm in order to compare outcomes, and to flag uncertainty in the event of discrepancies.

The method of FIG. 2 may be used for pre-processing before a coronary intervention. The method of FIG. 2 allows separation of both coronary and pulmonary vascular sub-trees surrounding the heart.

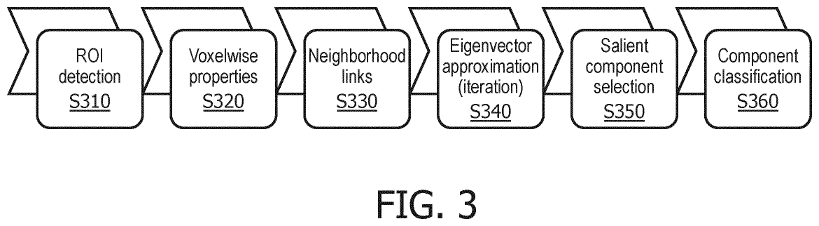

FIG. 3 illustrates another method for spectral clustering for detection of atypical cardiac coronaries, in accordance with a representative embodiment.

The method of FIG. 3 starts at S310 with detection of a region of interest which may be a volume of interest. The identification of the region of interest involves segmenting a cardiac chamber as a broad image volume of interest around the heart. The segmenting to detect the region of interest may be performed using model-based segmentation (MBS) or other machine learning-based semantic segmentations. A machine learning-based semantic segmentation may be performed by a deep convolutional neural network, for example.

The method of FIG. 3 treats all voxels of an image volume of interest as graph nodes. Each voxel is considered to be fully connected to its local neighbors, but with a continuous (rather than binary) linkage weight to each neighbor. The linkage weight depends on the vesselness-features, multi-spectral similarity, and radial and directional affinity.

At S320, properties are determined for each voxel. The properties may include characteristics of each voxel that may reflect the likelihood of the voxel representing a vessel in a coronary constellation. The determination at S320 may be performed as a vesselness filter response. A single-channel image may be generated containing vesselness filter-responses for each voxel in the volume of interest considering all spectral channels. The vesselness filter-responses may include magnitude, radius and direction estimation. The responses may be generated as functions/eigenvalues of the Hessian matrix of second derivatives, optionally with additional provisions against image noise.

In some embodiments, a vesselness filter may be implemented using a trained artificial intelligence model. The vesselness filter may be implemented as a machine learning component such as by using a convolutional neural network, instead of as an analytical filter function. The vesselness filter may be trained from a set of available data, such as from annotated coronaries.

At S330, neighborhood links are identified. Neighborhood links are a similarity measure computation in local neighborhoods of voxels in the volume of interest. For each pair of voxels within a limited neighborhood, a symmetric similarity measure s_ij between voxels i and j is computed, to quantify the strength of the link between those two voxels. The symmetric similarity measures may be defined as positive and semidefinite.

Approximate spectral clustering involves implicitly requiring the Laplacian matrix L or a variant such as “normalized” or “random walk” between all voxels in the volume of interest. The matrix may be given by L=D−S, where S=(s_ij) is the similarity matrix and D=diag(S1) is the (diagonal) degree matrix. Spectral clustering proceeds by computing the first several eigenvectors of the (very sparse) Laplacian matrix. In practice, these algorithms rely on matrix vector multiplications (MVMs) with the Laplacian matrix, such as power iteration, Lanczos and the like.

At S340, an iterative eigenvector approximation is performed. The iterative eigenvector approximation is an iterative identification of salient through-flow voxels and tree root voxels. These voxels stand out due to their most ‘influential’ roles in the graph of voxels.

In some embodiments, low ranking voxels may be excluded from iterations once identified. Exclusion of low ranking voxels may accelerate the processing in the method of FIG. 3.

At S350, salient components are selected. S350 involves a selection of subtrees. Separated local rank score peak locations may be selected and used as seeds for building subtrees from all ‘upstream’ voxels.

At S360, selected components are classified. Rule-based classification of subtrees may use heuristics, to be pulmonary vessels or coronaries. Classification as pulmonary vessels may be based on being embedded in pulmonary tissue and may use semantic segmentation (labeling). Classification as coronaries may be based on exhibiting terminals somewhere at the aorta and the myocardium.

The method of FIG. 3 is used for pre-processing/normalization and for vessel segment clustering. For pre-processing/normalization, standard vessel filters yield an uncalibrated response, depending on varying image characteristics such as contrast, noise and resolution. In contrast, the eigenvector of the Laplacian performed at S340 is a normalized vector, so that the voxel-wise response is determined by topology, flow, confluence, stenoses, etc. For vessel segment clustering, scattered vessel-filter responses are aggregated into natural clusters, representing vessel segments, which can then be classified and processed further by virtue of their global properties. The natural clusters are connected by flow-determined affinity. The size of the natural clusters increases with iterations.

The method of FIG. 2 and the method of FIG. 3 describe a global analytical algorithm with few parameters. No extensive machine learning training data is required, and this helps avoid the potential requirements that may accompany machine learning training data such as annotations, sampling, imaging protocol coverage, and regulatory efforts. Indeed, the analytical algorithm provided by the method of FIG. 2 and the method of FIG. 3 may be used to generate and/or accelerate semi-automated curation of training ‘ground truth’ for artificial intelligence algorithms.

The embodiments of FIG. 2 and FIG. 3 are primarily described with respect to spectral computerized tomography. However, other imaging modalities may use the spectral clustering described herein. Other imaging modalities include magnetic resonance imaging, ultrasound, single-photon emission computerized tomography, positron emission tomography and more. Additionally, the teachings herein are not limited to cardiac coronary vessels as the volumes of interest. Rather, the teachings herein are applicable other anatomical trees such as lung vessels trees of veins and arteries and for lung lobes, lobar bronchial airway trees, hepatic vessel trees and more.

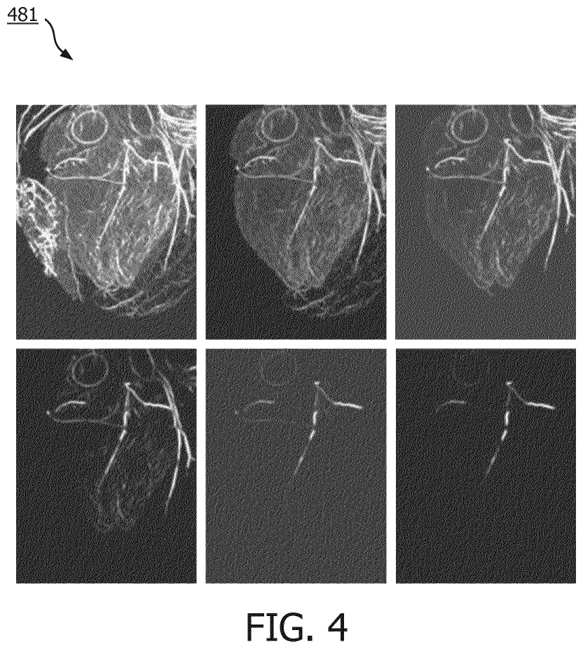

FIG. 4 illustrates an example of a power iteration for spectral clustering for detection of atypical cardiac coronaries, in accordance with a representative embodiment.

FIG. 4 illustrates an example of a power iteration on the Laplacian matrix in a cardiac volume of interest, shown as maximum projections perpendicular to the left-ventricle-long-axis. The exemplary graph consists of 6 677315 nodes (=length of eigenvector) with links in a 7×7×7 neighborhood, with a maximum of 171 links, and an average of 33 neighbor links per node. The top left panel shows all vesselness filter responses. The top middle panel shows components connected to the ascending aorta (as segmented by model-based segmentation, MBS). The top right panel and all three bottom panels show iterations, with connected components formed by scores above a threshold of 2×meanScore, and connected to the ascending aorta.

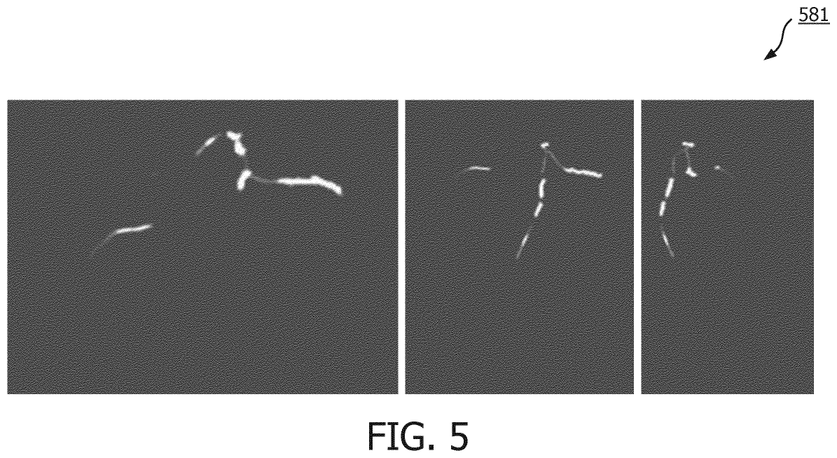

FIG. 5 illustrates an example of projections for spectral clustering for detection of atypical cardiac coronaries, in accordance with a representative embodiment.

FIG. 5 illustrates example axial, coronal, and sagittal maximum projections relative to left ventricular-long-axis of scores after 8 power iterations of clusters/components connected to the ascending aorta.

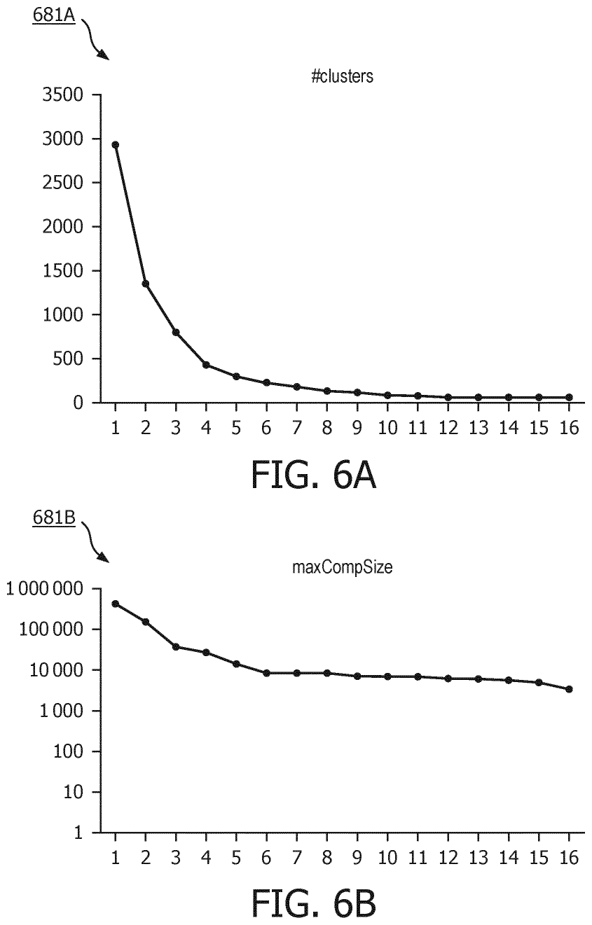

FIG. 6A illustrates a chart for a power iteration for spectral clustering for detection of atypical cardiac coronaries, in accordance with a representative embodiment. FIG. 6B illustrates a chart of maximum power sizes over iterations for the power iteration in FIG. 6A, in accordance with a representative embodiment.

In FIG. 6A, a power iteration on the Laplacian matrix in a cardiac volume of interest shows the decrease of connected components of scores above 2× meanScore. In FIG. 6B, maximum component size is also decreasing with iterations, as more graph nodes have vanishing scores, and only salient nodes remain significantly above (2×) the mean score.

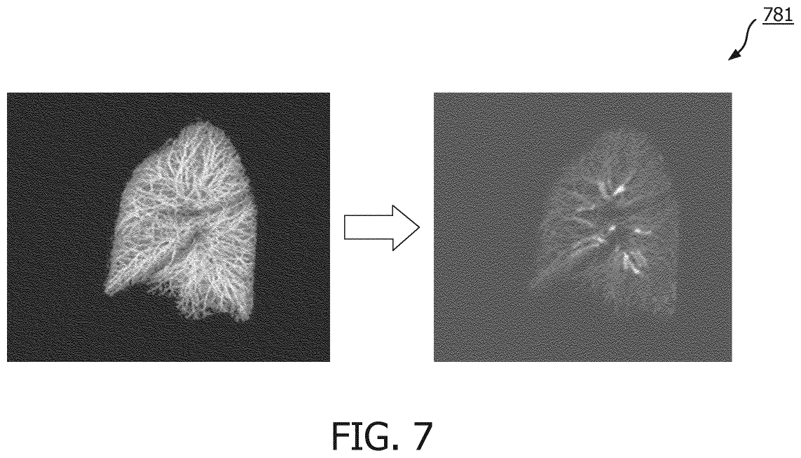

FIG. 7 illustrates an example application on pulmonary vessel trees in spectral clustering for detection of atypical cardiac coronaries, in accordance with a representative embodiment.

In FIG. 7, the left side shows dense voxel candidate input and the right side shows that, after iteration, the network rank of voxels is displayed as brightness, showing the most influential stumps of subtrees. On the right side of FIG. 7, the most influential stumps of subtrees correspond to the sagittal projection.

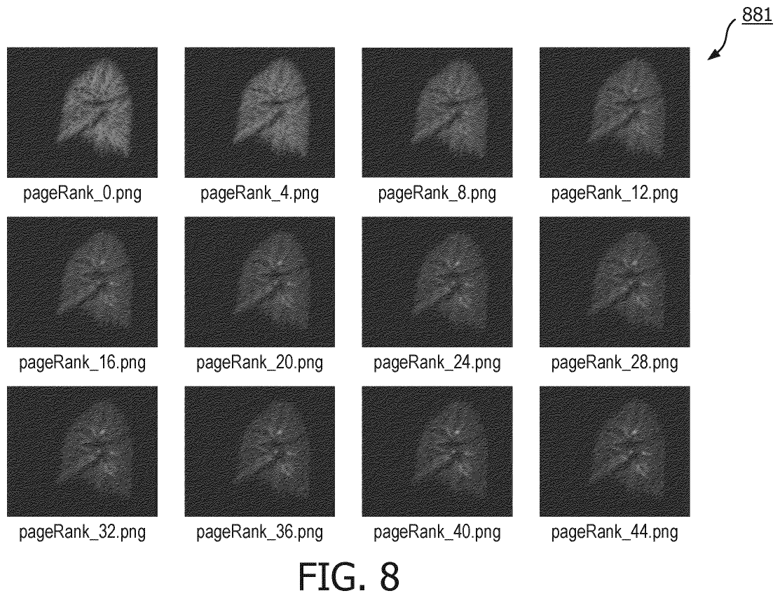

FIG. 8 illustrates an example diffusion process of evolving network ran of candidate voxels in spectral clustering for detection of atypical cardiac coronaries, in accordance with a representative embodiment.

The diffusion process in FIG. 8 is an example iteration of an evolving network rank of all candidate voxels, and is based on the sagittal projection as on the right side of FIG. 7.

FIG. 9 illustrates a computer system, on which a method for spectral clustering for detection of atypical cardiac coronaries is implemented, in accordance with another representative embodiment.

Referring to FIG. 9, the computer system 900 includes a set of software instructions that can be executed to cause the computer system 900 to perform any of the methods or computer-based functions disclosed herein. The computer system 900 may operate as a standalone device or may be connected, for example, using a network 901, to other computer systems or peripheral devices. In embodiments, a computer system 900 performs logical processing based on digital signals received via an analog-to-digital converter.

In a networked deployment, the computer system 900 operates in the capacity of a server or as a client user computer in a server-client user network environment, or as a peer computer system in a peer-to-peer (or distributed) network environment. The computer system 900 can also be implemented as or incorporated into various devices, such as a workstation that includes a controller, a stationary computer, a mobile computer, a personal computer (PC), a laptop computer, a tablet computer, or any other machine capable of executing a set of software instructions (sequential or otherwise) that specify actions to be taken by that machine. The computer system 900 can be incorporated as or in a device that in turn is in an integrated system that includes additional devices. In an embodiment, the computer system 900 can be implemented using electronic devices that provide voice, video or data communication. Further, while the computer system 900 is illustrated in the singular, the term “system” shall also be taken to include any collection of systems or sub-systems that individually or jointly execute a set, or multiple sets, of software instructions to perform one or more computer functions.

As illustrated in FIG. 9, the computer system 900 includes a processor 910. The processor 910 may be considered a representative example of a processor of a controller and executes instructions to implement some or all aspects of methods and processes described herein. The processor 910 is tangible and non-transitory. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The processor 910 is an article of manufacture and/or a machine component. The processor 910 is configured to execute software instructions to perform functions as described in the various embodiments herein. The processor 910 may be a general-purpose processor or may be part of an application specific integrated circuit (ASIC). The processor 910 may also be a microprocessor, a microcomputer, a processor chip, a controller, a microcontroller, a digital signal processor (DSP), a state machine, or a programmable logic device. The processor 910 may also be a logical circuit, including a programmable gate array (PGA), such as a field programmable gate array (FPGA), or another type of circuit that includes discrete gate and/or transistor logic. The processor 910 may be a central processing unit (CPU), a graphics processing unit (GPU), or both. Additionally, any processor described herein may include multiple processors, parallel processors, or both. Multiple processors may be included in, or coupled to, a single device or multiple devices.

The term “processor” as used herein encompasses an electronic component able to execute a program or machine executable instruction. References to a computing device comprising “a processor” should be interpreted to include more than one processor or processing core, as in a multi-core processor. A processor may also refer to a collection of processors within a single computer system or distributed among multiple computer systems. The term computing device should also be interpreted to include a collection or network of computing devices each including a processor or processors. Programs have software instructions performed by one or multiple processors that may be within the same computing device or which may be distributed across multiple computing devices.

The computer system 900 further includes a main memory 920 and a static memory 930, where memories in the computer system 900 communicate with each other and the processor 910 via a bus 908. Either or both of the main memory 920 and the static memory 930 may be considered representative examples of a memory of a controller, and store instructions used to implement some or all aspects of methods and processes described herein. Memories described herein are tangible storage mediums for storing data and executable software instructions and are non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The main memory 920 and the static memory 930 are articles of manufacture and/or machine components. The main memory 920 and the static memory 930 are computer-readable mediums from which data and executable software instructions can be read by a computer (e.g., the processor 910). Each of the main memory 920 and the static memory 930 may be implemented as one or more of random access memory (RAM), read only memory (ROM), flash memory, electrically programmable read only memory (EPROM), electrically erasable programmable read-only memory (EEPROM), registers, a hard disk, a removable disk, tape, compact disk read only memory (CD-ROM), digital versatile disk (DVD), floppy disk, blu-ray disk, or any other form of storage medium known in the art. The memories may be volatile or non-volatile, secure and/or encrypted, unsecure and/or unencrypted.

“Memory” is an example of a computer-readable storage medium. Computer memory is any memory which is directly accessible to a processor. Examples of computer memory include, but are not limited to RAM memory, registers, and register files. References to “computer memory” or “memory” should be interpreted as possibly being multiple memories. The memory may for instance be multiple memories within the same computer system. The memory may also be multiple memories distributed amongst multiple computer systems or computing devices.

As shown, the computer system 900 further includes a video display unit 950, such as a liquid crystal display (LCD), an organic light emitting diode (OLED), a flat panel display, a solid-state display, or a cathode ray tube (CRT), for example. Additionally, the computer system 900 includes an input device 960, such as a keyboard/virtual keyboard or touch-sensitive input screen or speech input with speech recognition, and a cursor control device 970, such as a mouse or touch-sensitive input screen or pad. The computer system 900 also optionally includes a disk drive unit 980, a signal generation device 990, such as a speaker or remote control, and/or a network interface device 940.

In an embodiment, as depicted in FIG. 9, the disk drive unit 980 includes a computer-readable medium 982 in which one or more sets of software instructions 984 (software) are embedded. The sets of software instructions 984 are read from the computer-readable medium 982 to be executed by the processor 910. Further, the software instructions 984, when executed by the processor 910, perform one or more steps of the methods and processes as described herein. In an embodiment, the software instructions 984 reside all or in part within the main memory 920, the static memory 930 and/or the processor 910 during execution by the computer system 900. Further, the computer-readable medium 982 may include software instructions 984 or receive and execute software instructions 984 responsive to a propagated signal, so that a device connected to a network 901 communicates voice, video or data over the network 901. The software instructions 984 may be transmitted or received over the network 901 via the network interface device 940.

In an embodiment, dedicated hardware implementations, such as application-specific integrated circuits (ASICs), field programmable gate arrays (FPGAs), programmable logic arrays and other hardware components, are constructed to implement one or more of the methods described herein. One or more embodiments described herein may implement functions using two or more specific interconnected hardware modules or devices with related control and data signals that can be communicated between and through the modules. Accordingly, the present disclosure encompasses software, firmware, and hardware implementations. Nothing in the present application should be interpreted as being implemented or implementable solely with software and not hardware such as a tangible non-transitory processor and/or memory.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

Accordingly, spectral clustering for detection of atypical cardiac coronaries enables identification, segmentation and development of cardiac coronaries which may otherwise be missed by machine learning models looking for typical cardiac coronaries. The teachings herein may result in an ability to diagnose and treat cardiac events which otherwise would not be diagnosed and treated.

Although spectral clustering for detection of atypical cardiac coronaries has been described with reference to several exemplary embodiments, it is understood that the words that have been used are words of description and illustration, rather than words of limitation. Changes may be made within the purview of the appended claims, as presently stated and as amended, without departing from the scope and spirit of spectral clustering for detection of atypical cardiac coronaries in its aspects. Although spectral clustering for detection of atypical cardiac coronaries has been described with reference to particular means, materials and embodiments, spectral clustering for detection of atypical cardiac coronaries is not intended to be limited to the particulars disclosed; rather spectral clustering for detection of atypical cardiac coronaries extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A method of spectral clustering, comprising:
obtaining a set of coronary image data comprising voxels of a volume of interest around a heart;
processing the voxels to rank the voxels for likelihood of characterizing a vessel;
identifying similarities between pairs of voxels to quantify strength of a link between the voxels in each pair of voxels;
spectral clustering the voxels based on the likelihood of characterizing a vessel and strength of links between the pairs of voxels;
selecting at least one subtree of voxels based on the spectral clustering;
classifying each subtree based on the set of coronary image data; and
reconstructing a representation of at least one vessel in the volume of interest as a first reconstruction to include at least one subtree classified based on the set of coronary image data.

2. The method of claim 1, further comprising:
generating a second reconstruction in the volume of interest without performing the spectral clustering and selecting; and
comparing the first reconstruction and the second reconstruction to identify atypical coronary constellations from the at least one subtree of voxels selected based on the spectral clustering.

3. The method of claim 1, wherein the processing of the voxels includes filtering the voxels based on image properties expected of vessels.

4. The method of claim 3, wherein the similarities are identified based on the image properties expected of vessels.

5. The method of claim 1, wherein the processing of the voxels with power iterations, the identification of similarities, and the spectral clustering are performed for voxels on a 1-to-1 basis by cores of a graphical processor.

6. The method of claim 1, wherein the similarities are identified based on proximity of coordinates of each voxel in a pair of voxels and directionality of each voxel in a pair of voxels.

7. The method of claim 1, wherein the likelihood of a voxel characterizing a vessel is determined based on a vesselness filter applied to the voxel.

8. The method of claim 1, wherein the similarities are identified for pairs of proximate voxels and are not identified for pairs of remote voxels.

9. The method of claim 1, wherein the spectral clustering is performed based on a matrix of the voxels in the volume of interest and using power iteration clustering.

10. A system for spectral clustering, comprising:
a memory configured to store instructions; and
a processor configured to execute the instructions, wherein, when executed by the processor, the instructions cause the processor to:
obtain a set of coronary image data comprising voxels of a volume of interest around a heart;
process the voxels to rank the voxels for likelihood of characterizing a vessel;
identify similarities between pairs of voxels to quantify strength of a link between the voxels in each pair of voxels;
spectral cluster the voxels based on the likelihood of characterizing a vessel and strength of links between the pairs of voxels;
select at least one subtree of voxels based on the spectral clustering;
classify each subtree based on the set of coronary image data; and
reconstruct a representation of at least one vessel in the volume of interest as a first reconstruction to include at least one subtree classified based on the set of coronary image data.

11. The system of claim 10, wherein the processing of the voxels includes filtering the voxels based on image properties expected of vessels.

12. The system of claim 11, wherein the similarities are identified based on the image properties expected of vessels.

13. The system of claim 10, wherein the processing of the voxels with power iterations, the identification of similarities, and the spectral clustering are performed for voxels on a 1-to-1 basis by cores of a graphical processor.

14. The system of claim 10, wherein the similarities are identified based on proximity of coordinates of each voxel in a pair of voxels and directionality of each voxel in a pair of voxels.

15. The system of claim 10, wherein the likelihood of a voxel characterizing a vessel is determined based on a vesselness filter applied to the voxel.

16. The system of claim 10, wherein the similarities are identified for pairs of proximate voxels and are not identified for pairs of remote voxels.

17. The system of claim 10, wherein the spectral clustering is performed based on a matrix of the voxels in the volume of interest and using power iteration clustering.

18. A non-transitory computer-readable storage medium having stored instructions which when executed by a processor, causes the processor to:
obtain a set of coronary image data comprising voxels of a volume of interest around a heart;
process the voxels to rank the voxels for likelihood of characterizing a vessel;
identify similarities between pairs of voxels to quantify strength of a link between the voxels in each pair of voxels;
spectral cluster the voxels based on the likelihood of characterizing a vessel and strength of links between the pairs of voxels;
select at least one subtree of voxels based on the spectral clustering;
classify each subtree based on the set of coronary image data; and
reconstruct a representation of at least one vessel in the volume of interest as a first reconstruction to include at least one subtree classified based on the set of coronary image data.

19. The non-transitory computer-readable storage medium of claim 18, wherein the similarities are identified based on proximity of coordinates of each voxel in a pair of voxels and directionality of each voxel in a pair of voxels.

20. The non-transitory computer-readable storage medium of claim 18, wherein the processing of the voxels includes filtering the voxels based on image properties expected of vessels.
